(19) United States
(12) Patent Application Publication
(10) Pub. No.: US 2009/0105670 A1
(43) Pub. Date: Apr. 23, 2009

(54) THIN FILM WOUND COVER, SUCTION ASSISTED WOUND TREATMENT SYSTEM USING THE SAME, METHOD OF USING THE THIN FILM WOUND COVER AND METHOD OF MAKING THE SAME

(75) Inventors: Lauren E. Bentley, Coatesville, PA (US); Allison L. Lloyd, Friendswood, TX (US); Christopher L. Radl, Malvern, PA (US); Trevor G. Smith, Phoenixville, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: BOEHRINGER TECHNOLOGIES, L.P., Norristown, PA (US)

(21) Appl. No.: 12/248,345

(22) Filed: Oct. 9, 2008

Related U.S. Application Data
(60) Provisional application No. 61/000,055, filed on Oct. 23, 2007.

(51) Int. Cl. A61F 13/02; A61M 1/00; B32B 37/12

(52) U.S. Cl. 604/290; 602/57; 604/313; 156/289

(57) ABSTRACT

A composite structure, a suction-assisted wound treatment system including the composite structure and a method of making the same is disclosed. The composite structure includes a cover, a stiffener, and a releasable liner. The cover is a thin flexible film having an, upper surface, an undersurface, a principal portion, a pair of marginal edges and an adhesive on the undersurface. The stiffener is releasably secured to the upper surface of the principal portion of the cover and includes a handle which is more rigid than the principal portion of the stiffener. The handle, forms a portion of the undersurface of the stiffener and defines a finger space between it and portion of the cover disposed therebelow. The liner includes at least one section releasably secured to the adhesive of the cover. The stiffener and the handle are removable as a unit from the cover.

### CROSS-REFERENCE TO RELATED APPLICATIONS

This application claims priority from U.S. Provisional Application 61/000,055 filed on Oct. 23, 2007, entitled Thin Film: Wound Cover, which is assigned to the same assignee as this application and whose disclosure is incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### FIELD OF THE INVENTION

This invention relates generally to medical covers and more particularly to adhesive polymeric dressings or covers which can be readily applied to a patient for suction assisted wound care or other medical applications.

### BACKGROUND OF THE INVENTION

Conformable polymeric dressings have gained wide acceptance for use as protective, layers on wounds to manage or otherwise facilitate healing by establishing a moist environment that is isolated from the ambient surroundings. Such dressings are typically formed of a very thin transparent polymeric film having a self-sticking adhesive under-surface for attachment to the patient's body, e.g., the patient's skin, contiguous with the wound. While, such dressings offer various advantages over traditional absorbent dressings, such as faster healing, enhanced autolysis of necrotic tissue and reduced patient discomfort, they are somewhat difficult to handle and apply due to their extreme thinness and the presence of the self-sticking adhesive. Moreover, such thin adhesive covers are incapable of supporting themselves. Thus, when handled, they start to wrinkle. Because of the adhesive, once they start to wrinkle before application, they can become unusable. Also, since the adhesive is very sticky it can stick to gloves easily unless it is covered. To that end, many adhesive thin film dressings are supplied with a releasable protective liner covering the adhesive to protect the adhesive and facilitate handling. However, once the liner has been removed, the adhesive coated film can still wrinkle and adhere to itself, interfering with the smooth, aseptic application of the dressing to a patient's skin unless some other means are provided to prevent such undesirable action. Thus, various delivery systems have been proposed in the patent literature and some are commercially available to address these problems.

For example in. U.S. Pat. No. 4,485,809 (Dellas) there is disclosed a film dressing having a release sheet attached to the dressing. A central region of the film, defined by perforation lines, is applied to the patient. There are cut lines in the release sheet which are parallel to but spaced outside the perforations in the dressing to allow the release sheet to be removed, the adhesive portion of the film to be applied to the patient and the exterior portion of both the release sheet and the film to be removed.

In U.S. Pat. No. 4,614,183 (McCracken et al.) there is disclosed a polymeric film dressing whose adhesive surface is covered by a release paper liner formed in three laterally disposed sections to facilitate application to the dressing of the wound at the wound site. The central section is arranged to be removed and the dressing grasped by the two side sections to place it in the desired position and then the side sections removed to complete the securement of the dressing to the wound.

In U.S. Pat. No. 4,600,001 (Gilman) there is disclosed a wound dressing and delivery laminate composite in the form of contiguously oriented and coplanar layers. A centrally-disposed wound dressing layer of polyurethane is in releasable adhesive contact with a release liner layer. The opposite side of the wound dressing layer is in the form of a non-adhesive surface that is releasably heat laminated to a delivery layer of ethylene vinyl acetate (EVA). A pair of tape strips are secured to the upper surface of the dressing layer on opposite edges, with the tape strips being bounded by perforated lines in that layer. In use the release liner layer is separated from the adhesive surface of the dressing layer by peeling it off. The remaining adherent wound dressing layer and delivery layer of the composite are then positioned over the wound and applied thereto by contact adhesion. The delivery layer is then peeled off of the adjacent contiguous adhering surface of the wound dressing layer at a corner thereof. Finally, the tape strips are removed from the dressing layer by tearing them off along the perforated lines.

In U.S. Pat. No. 5,018,516 (Gilman) there is disclosed a delivery system for a wound dressing. The dressing is an elastomeric, e.g., polyurethane, film having a front surface, a back surface, an adhesive on thee front surface, a first end margin, and a second opposed end margin. The system includes a release sheet releasably attached to and covering the adhesive on the film and a tab member secured to the back surface of the film adjacent to the first end margin. A support sheet is releasably attached to the back surface of the film, with the support sheet having a first end margin located adjacent to the tab member and being free of attachment to the tab member, and a second end margin located adjacent to the second end margin of the film.

In U.S. Pat. No. 5,437,622 (Carion) there is disclosed a transparent adhesive dressing of synthetic material having reinforced starter cuts. The dressing contains three layers, namely, a sheet of flexible film made of polyurethane having an adhesive face, a protective sheet of backing covering the adhesive face, and a sheet of less flexible material made of polyethylene. A starter cut passes through at least two of the three layers. The starter cut is protected by a reinforcing strip disposed on the sheet of the less flexible film.

In U.S. Pat. No. 6,685,682 (Heinecke et al.) there is disclosed a carrier delivered dressing which has a conformable backing with a pressure sensitive adhesive coated on a bottom face and a low adhesion coating on a top face. The backing is supported during shipping and handling by a liner attached to the adhesive and a removable heat sealed carrier attached to the top face of the backing.

In United States Published Application No. 2007/0156075 (Heinecke) there is disclosed a carrier delivered dressing which has a conformable backing with a pressure sensitive adhesive coated on a bottom face and removable carrier attached to the top face of the backing. A bond block material is positioned between thee backing and the carrier. A cut line traverses both the carrier and the bond block material to form a tab.

In EPO Publication No. 0 051 935 there is disclosed pressure-sensitive adhesive-coated relatively thin, conformable polymeric film with a releasable layer attached to the surface of the film opposite to that containing the adhesive, which layer is attached in a more tenacious manner than a release liner covering the adhesive of the film.

Examples of various polymeric film materials that are commercially available for application to wounds are sold under the names POLYSKIN II™ by Tyco Healthcare Group, Mansfield, Mass., TEGADERM™ by 3M Company, St. Paul, Mnn., BIOCLUSIVE™ by Johnson & Johnson Company, New Brunswick, N.J.), OP-SITE™ by T. J. Smith & Nephew, Hull, England and VACUUM ASSISTED CLOSURE® drape by KCI, Inc. of San Antonio, Tex.

The POLYSKIN II™ cover of Tyco Healthcare Group appears to be constructed in accordance with U.S. Pat. No. 5,018,516 (Gilman) and is somewhat similar to U.S. Pat. No. 4,600,001. (Gilman). To that end, it basically comprises a composite structure including a dressing layer in the form of a polyurethane film having an adhesive on its undersurface. A carrier layer formed of EVA is releasably secured to the top surface of the dressing layer. The carrier layer includes a first handle strip at one of its marginal edges and a second handle strip at the opposite marginal edge. A common liner sheet is releasably secured to the adhesive on the underside of the dressing layer. The liner sheet extends beyond the marginal edge of the first handle strip of the carrier layer. A paper tape strip is secured to the top surface of the dressing layer directly under the second handle strip. The dressing layer is perforated along the paper tape strip. In use the cover is applied by removing the liner sheet while holding carrier layer by the first handle strip. The combined dressing and carrier layers can then be held by the two handle strips of the carrier layer to apply that combination to the wound so that the adhesive on the underside of the dressing layer is brought into engagement with the wound bed. The carrier can then be removed by separating the second handle strip from the tape strip disposed thereunder and then holding the tape strip in place with a finger. The carrier sheet can then be peeled off of the polyurethane layer by grasping its second handle strip and pulling on the handle strip. Finally, the tape strip can be removed from the polyurethane dressing layer by tearing them off along the perforated line.

The VACUUM ASSISTED CLOSURE® drape of KCI, Inc. basically comprises a composite structure including a dressing layer in the form of a polyurethane film secured to a polyethylene carrier layer, so that the carrier forms the top layer of the composite structure. The underside of the polyurethane layer includes an adhesive which is covered by three removable liner sheets, somewhat similar in construction to that disclosed in U.S. Pat. No. 4,614,183 (McCracken et al.), A pair of handle strips is secured along opposed marginal edges of the urethane film, i.e., the strips are adhesively secured to the polytethane layer by the adhesive on the underside of that layer. The polyurethane layer is perforated along the handle strips. In use the liner sheets are removed to secure the polyurethane film to the wound. Then the polyethylene carrier layer is removed. Finally, the handle strips are removed from the polyurethane layer by tearing them off along the perforated lines.

While the devices of the aforementioned patents and commercially available products are generally suitable for their intended purposes, they nevertheless leave much to be desired from one or more of the standpoints of: disturbance of the wound during application of the cover, effectiveness, ease of use, simplicity of construction, cost and ease of manufacture.

All references cited and/or identified herein are specifically incorporated by reference herein.

### SUMMARY OF THE INVENTION

In accordance with one aspect of the invention there is provided a composite structure comprising a cover, a stiffener, and a releasable liner. The cover is arranged for use on a wound of a patient and is formed of a very thin, flexible film, e.g., polyurethane, having an upper surface, an undersurface, a principal portion, a pair of opposed marginal edges and an adhesive on the undersurface of the film. The stiffener comprises a sheet, e.g., an EVA sheet, having an upper surface, an undersurface, a principal portion, a pair of opposed marginal edges and at least one handle. The at least one handle is more rigid than the principal portion of the stiffener and forms a portion of the undersurface of the stiffener contiguous with a marginal edge of the stiffener. The stiffener is disposed over the cover, with the undersurface of the stiffener at the principal portion of the stiffener being releasably secured to the upper surface of the cover at the principal portion of the cover. The at least one handle defines between it and the marginal edge of the cover disposed therebelow a finger space in which a finger of a user can be inserted to separate the stiffener from the cover. The releasable liner comprises at least one section releasably secured to the adhesive to protect the adhesive and being removable when desired to expose the adhesive, whereupon the cover can be adhesively secured over the wound by the adhesive. The stiffener and the at least one handle are removable as a unit from the cover.

In accordance with another aspect of this invention there is provided a composite structure comprising a cover and a stiffener. The cover is arranged for use on a wound of a patient and is formed of a very thin, flexible film, e.g., polyurethane, having an upper surface, an undersurface, at least one marginal edge portion, a principal portion and an adhesive on the undersurface of the film. The at least one marginal edge portion of the cover and the principal portion of the cover are of essentially the same flexibility as each other. The stiffener comprises a sheet, e.g., an EVA sheet, having an upper surface, an undersurface, at least one marginal edge portion and a principal portion. The stiffener is disposed over the cover with the undersurface of the stiffener at the principal portion of the stiffener being releasably secured to the upper surface of the cover at the principal portion of the cover. The at least one marginal edge portion of the stiffener is separated from the at least one marginal edge portion of the cover disposed therebelow by a finger space.

In accordance with another aspect of this invention there is provided a suction assisted wound care system comprising, a source, of suction, a coupling member, and a composite structure. The composite structure comprises a cover adapted to be adhesively secured over a wound of a patient to create a confined space to which a vacuum can be applied, a stiffener and a releasably securable liner. The coupling member, e.g., a suction tube attachment device, is arranged to be coupled to the source of suction. e.g., a portable vacuum pump, for applying suction to the confined space. The cover is formed of a very thin, flexible film, e.g., polyurethane, having an upper surfacer an undersurface, a principal portion, at least one marginal edge and an adhesive on the undersurface of the film arranged to secure the cover to the patient. The stiffener comprises a sheet of material, e.g., EVA, having an upper surface, an undersurface, a principal portion, at least one marginal edge and at least one handle. The at least one handle forms a portion of the undersurface of the stiffener contiguous with the at least one marginal edge of the stiffener. The stiffener is disposed over the cover, with the undersurface of the stiffener at the principal portion of the stiffener releasably secured to the upper surface of the cover at the principal portion of the cover. The at least one handle defines between it and the at least one marginal edge-portion of the cover disposed therebelow a finger space in which a finger of a user can be inserted. The releasable liner comprises at least one section releasably secured to the adhesive to protect the adhesive and is removable when desired to expose the adhesive, whereupon the cover can be adhesively secured over the wound by the adhesive. The stiffener and the at least one handle are removable as a unit from the cover.

In accordance with one exemplary embodiment of the composite structure of this invention and the suction assisted wound care system of this invention, the at least one handle of the composite structure comprises a strip of adhesive tape.

The suction assisted wound care system of this invention may also make use of a wound packing having a wound contact surface that is used under the cover so that it is located within the confined space, with its wound contact surface engaging the wound.

In accordance with another aspect of this invention a method of providing suction assisted wound treatment to a wound of a patient is provided. That method comprises providing a composite structure like that set forth above, removing the releasable liner from the cover to expose the adhesive and applying the cover over the wound to produce a confined space to which suction can be applied and grasping the at least one handle of the composite structure via the finger space to remove the stiffener and the at least one handle as a unit from the cover. Suction can then be applied to the confined space from a source of suction. If desired, a wound packing having a wound contact surface can be located within the confined space, with its wound contact surface engaging the wound.

In accordance with another aspect of this invention a general method for treating a wound of a patient is provided. That method comprises providing a composite structure like that set forth above, removing the releasable liner from the cover to expose the adhesive and applying the cover over the wound and grasping the at least one handle of the composite structure via the finger space to remove the stiffener and the at least one handle as a unit from the cover. If desired, a wound packing having a wound contact surface can be located within the confined space, with its wound contact surface engaging the wound.

In accordance with another aspect of this invention a method for making a composite structure including a cover for adhesive application to the body of a patient is provided. That method entails providing a cover formed of a very thin, flexible film having an upper surface, an undersurface, a principal portion, at least one marginal edge and an adhesive on the undersurface of the cover arranged to secure the cover to the body of the patient. A stiffener is also provided. The stiffener comprises a sheet having an upper surface, an undersurface, a principal portion, at least one marginal edge. A strip is provided on the undersurface of the stiffener at the at least one marginal edge of the stiffener to form a handle thereat. The at least one handle is stiffer than the principal portion of the stiff. The stiffener and the cover are juxtaposed to releasably secure the undersurface of the stiffener at the principal portion of the stiffener to the upper surface of the cover at the principal portion of the cover.

In accordance with one preferred aspect of the method of making the composite structure, the undersurface of the stiffener at the principal portion of the stiffener is thermally releasably secured to the upper surface of the cover at the principal portion of the cover.

In accordance with another preferred aspect of the method of making the composite structure, the cover is provided as a continuous web on a waste liner. The liner sheet is applied in the form of a continuous web of material, e.g., three continuous webs, to the adhesive on the undersurface of the cover. The stiffener is also provided as a continuous web of sheet material.

### DESCRIPTION OF THE DRAWING

FIG. 1 is an exploded isometric view of a suction assisted wound care system incorporating one exemplary embodiment of a composite structure including a film cover constructed in accordance with this invention;

FIG. 2 is an isometric view of the underside of one of the components shown in FIG. 1, namely, the wound packing to show its wound contact surface;

FIG. 3 is a sectional view of the composite structure shown in FIG. 1;

FIG. 4 is an enlarged isometric view of the composite structure of FIGS. 1 and 3;

FIG. 5 is a sectional view, similar to FIG. 3, but showing an alternative exemplary embodiment of a composite structure of this invention;

FIG. 6 is a sectional view, similar to FIGS. 3 and 5, but showing another alternative exemplary embodiment of a composite structure of this invention;

FIG. 7 is a sectional view, similar to FIGS. 3, 5 and 6, but showing still another alternative exemplary embodiment of a composite structure of this invention;

FIG. 8 is a sectional view, similar to FIGS. 3, 5-7, but showing yet another alternative exemplary embodiment of a composite structure of this invention; and

FIG. 9 is an illustration of one exemplary manner of making composite structures of this invention.

### DETAILED DESCRIPTION OF THE PREFERRED EMBODIMENT

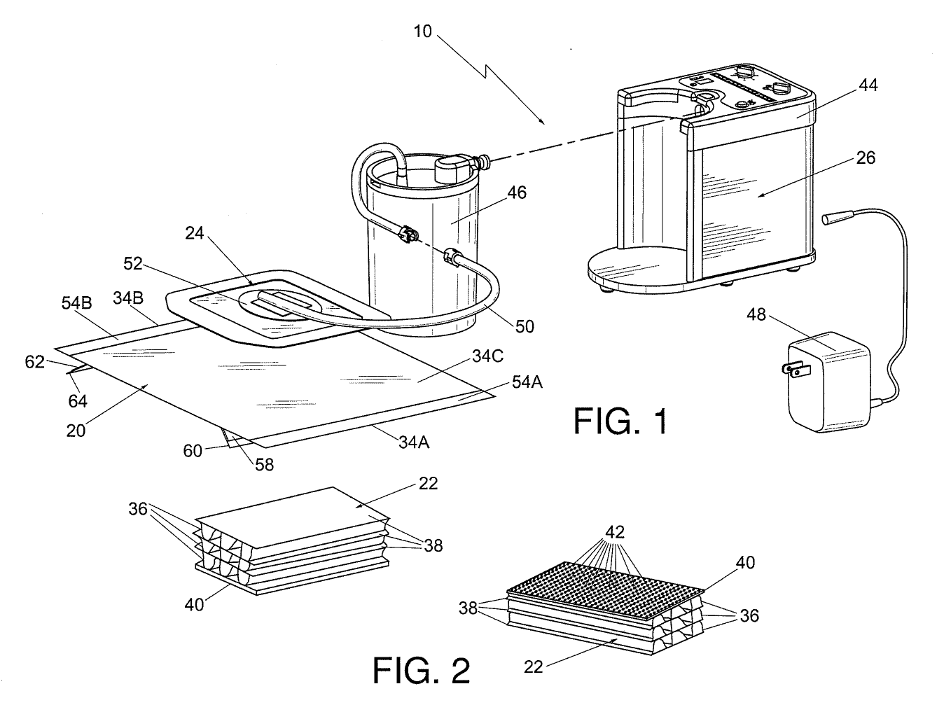

Referring now to the various figures of the drawing wherein like reference characters refer to like parts, there is shown in FIG. 1 a suction assisted (negative pressure) wound treatment system 10 constructed in accordance with an aspect of this invention and making use of a composite structure 20 (to be described shortly) including a wound cover that is also constructed in accordance with this invention. Before describing the system 10 and the composite structure it should be noted that aspects of the subject invention can be used with other suction assisted or negative pressure wound; treatment systems or devices and the composite structure 20 can be used for other applications than suction assisted wound treatment, e.g., as a general wound cover, surgical drape, etc.

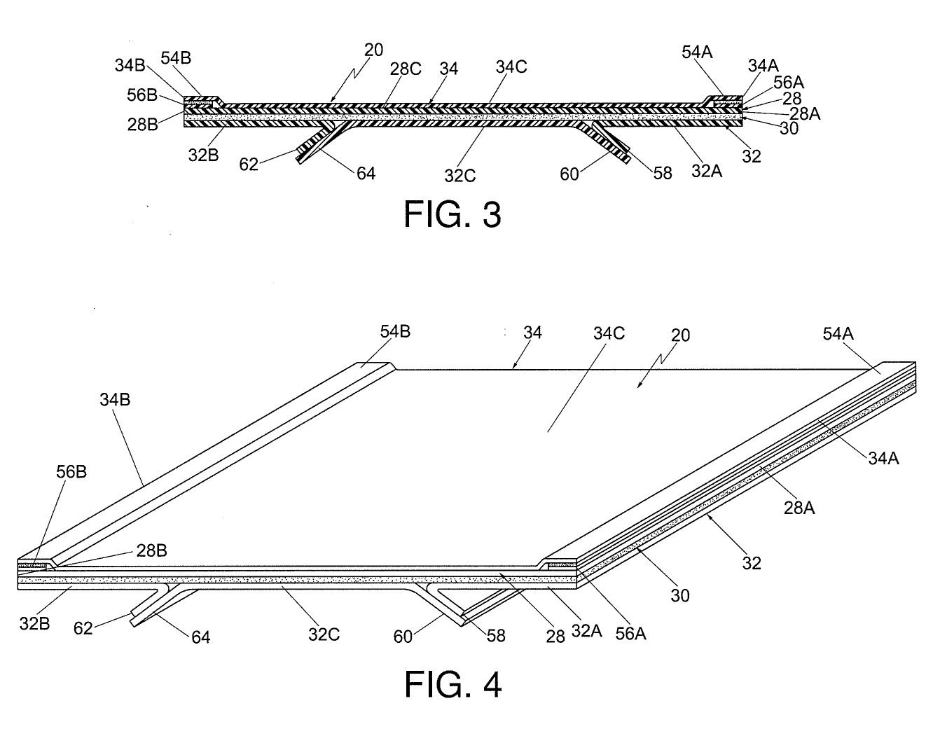

As seen in FIG. 1 the system 10 basically comprises the previously mentioned composite structure 20, a wound packing 22, a suction tube attachment device 24, and a portable pump and wound monitoring unit 26. The composite structure will be described in considerable detail shortly. Suffice it for now to state that as best seen in FIGS. 3 and 4 the composite structure 20 basically comprises a conformable cover 28 including an adhesive 30 on its undersurface for securement to the wound of a patient, a releasably securable liner 32 and a releasably securable stiffener 34. The releasably securable liner 32 and the stiffener 34 cooperate to facilitate the adhesive securement of the cover to the wound, as will be described in detail later.

Once the cover 28 is adhesively secured to the wound of the patient a confined space is created under the cover contiguous with the wound to which negative pressure or suction can be applied to treat or otherwise facilitate healing of the wound. Moreover, for many wound treatment applications it is desirable to make use of a wound packing under the cover and in contact with the wound. The wound packing 22 shown in FIG. 1 is exemplary of one particularly suitable and effective packing suitable for that purpose, although others can be used. Thus, in accordance with one preferred embodiment of the system 10 of this invention, the wound packing 22 is preferably constructed in accordance with the teachings of U.S. patent application Ser. No. 10/981,119, filed on Nov. 4, 2004, entitled Wound Pacing Material For Use With Suction, published as 2005209574 on Sep. 22, 2005, which is assigned to the same assignee as this invention and whose disclosure is incorporated by reference herein. In the interest of brevity all of the details of the construction and operation of the wound packing 22 will not be reiterated herein. Suffice it, to state that the wound packing 22 basically comprises at least one and preferably more undulating sheets 36 secured together by interposed generally planar sheets 38 to form a permeable corrugated structure like shown in FIGS. 1 and 2. The sheets 36 and 38 are permeable and non-absorbent, e.g., are preferably formed of a non-woven, synthetic, biocompatible polymer such as polyolefins, polymides and polyester. A generally planar sheet 40, also of a similar permeable, non-absorbent material, forms the distal side of the packing 22. The outer surface of the sheet 40 forms the wound contact surface, i.e., the surface that will be in engagement with the wound when the packing is in place under the cover 28.

In accordance with one preferred embodiment of the system 10 of this invention, the wound contact sheet 40 and its outer surface is constructed in accordance with the teachings of U.S. patent application Ser. No. 10/982,346, filed on Nov. 5, 2004, entitled Wound Contact Device, published as 2005/0228329 on Oct. 13, 2005, and U.S. patent application Ser. No. 11/825,397, filed on Jul. 6, 2007, entitled Growth Stimulating Wound Dressing With Improved Contact Surfaces, published as 2008/0177253 on Jul. 24, 2008, each of which is also assigned to the same assignee as this invention and whose disclosures are incorporated by reference herein. In the interest of brevity the details of the construction, and operation of the wound contact surface will not be reiterated herein. Suffice it to say that the sheet 40 is formed of a permeable non-absorbent material like that described above and having an outer surface that is in the form of a thin film, e.g., a material chosen from the group consisting of polyester film, cellulose acetate film, and vinyl film. The wound contact surface includes a multitude of small dimple recesses or voids 42 in it and extending slightly into the sheet 40. These voids are resistant to collapse when the packing is subjected to suction explained in detail in the aforementioned co-pending patent applications.

The pump and wound monitoring unit 26 is constructed in accordance with the teachings of U.S. patent application Ser. No. 11/268,212, filed on Nov. 7, 2005, entitled System for Treating A Wound With Suction and Method of Detecting Loss of Suction, published as 2007/0016152 on Jan. 18, 2007, and U.S. patent application Ser. No. 11/786,475, filed on Apr. 12, 2007, entitled Pump System For Negative Pressure Wound Therapy, published as 2007/0219532 on Sep. 20, 2007, each of which is also assigned to the same assignee as this invention and whose disclosures are incorporated by reference herein. In the interest of brevity the details of the construction and operation of the pump and wound monitoring unit 26 will not be reiterated herein. Suffice it for now to state that the unit 26 includes a pump that is arranged to produce negative pressure or suction and which is conveyed to the confined space under the cover 28 by the tube attachment device 24 when the cover is in place on the wound.

The unit 26 basically comprises a portable suction pump (not shown) with a housing 44. Control circuitry (not shown) is also located in the housing 44. A reservoir or receptacle or waste collection canister 46 is releasably securable to the housing, i.e., the housing has a recess or cavity for receipt of the canister 46. The pump is configured to provide controlled levels of suction to the wound and has a flow rate monitor to indicate abnormal operating conditions. The pump runs on wall power via a suitable power converter 48. The control circuitry controls the operation of the pump, e.g., ensures that negative pressure is applied when desired, and also provides other functions, e.g., alarms in the event of malfunctions in the system. The receptacle 46 is arranged to receive fluid from the wound, e.g., blood and other wound exudates. To that end, the receptacle 46 is in fluid communication with the suction tube attachment device 24 via a conduit 50.

The suction tube attachment device 24 is constructed in accordance with the teachings of U.S. patent application Ser. No. 11/181,128, filed on Jul. 14, 2005, entitled Tube Attachment Device for Wound Treatment, published as 2006/0100586 on May 11, 2006, which is also assigned to the same assignee as this invention and whose disclosure is incorporated by reference herein. In the interest, of brevity all of the details of the construction and operation of the device 24 will not be reiterated herein. Suffice it for now to state that the device 24 basically comprises the heretofore identified conduit 50 and a patch member 52 having an adhesive on its undersurface. The conduit 50 is in fluid communication with an internal passageway or aperture (not shown) in the patch member 52. The patch member is arranged to be secured by its adhesive layer to the top surface of the cover 28 after the cover has been secured in place over the wound. The aperture in the patch member 52, which is in fluid communication with the conduit 48, is also in fluid communication with an aperture (not shown) made in the cover 28 so that the aperture in the patch member will be in fluid communication with the confined space established under the cover 28 when the cover is secured to the wound. The aperture in the cover may be preformed or may be created by the user at the time of securement of the tube attachment device to the cover. The conduit 50 is connected to the canister 46 and is in fluid communication with the interior thereof. The interior of the canister is also in fluid communication with the inlet of the pump via a coupling (not shown). Thus, when the pump is operated it establishes negative pressure in the canister 46 and in the confined space under the cover 28, whereupon wound exudates are enabled to flow out of that confined space through the suction tube device 24 and into the canister 46 where they are collected for subsequent disposal.

It should be pointed out at this juncture that the composite structure of this invention can be used with other suction assisted wound treatment systems, such as those disclosed and claimed in U.S. patent application Ser. No. 10/663,226, filed on Sep. 16, 2003, entitled Device For Treating A Wound, published as 2004/0064132 on Apr. 1, 2004, and U.S. patent application Ser. No. 11/226,505, filed on Sep. 14, 2005, entitled Apparatus and Method For Suction-Assisted Wound Healing, published as 2006/0025727 on Feb. 2, 2006, both of which applications are also assigned to the same assignee as this invention and whose disclosures are incorporated by reference herein. Further still, the composite structure of this invention can be used with a tunnel dressing for a wound, that tunnel dressing being constricted in accordance with U.S. patent application Ser. No. 11/986,941, filed on Nov. 27, 2007, entitled Tunnel Dressing For Use In Negative Pressure Wound Therapy, published as 2008/0132819 on Jun. 5, 2008, which application is also assigned to the same assignee this invention and whose disclosure is incorporated by reference herein.

The details of the construction of the cover 28 and the other portions of the composite structure 20 will be described with particular reference to FIGS. 3-5. Those other portions of the composite structure, namely, the releasably securable liner 32 and the stiffener 34 can be thought of as a delivery system that is adapted to facilitate the transport of the cover 28 to the desired position over the wound and to facilitate its easy, effective and trouble-free securement to the wound.

The cover 28 is a conformable sheet, e.g., a polymeric film, having a pair of marginal side edges 28A and 28B, a central or principal portion 28C, an upper surface and a lower or undersurface having the adhesive 30 is disposed thereacross. The cover 28 with its adhesive 30 is of the same flexibility across its entire area. The cover with its adhesive undersurface can be of any suitable material for use for application to a wound for suction assisted wound care. One particularly suitable material is polyurethane film having a thickness in the range of 0.0005 inches-0.002 inches, with a preferred thickness of approximately 0.0008 inches. The film is moisture vapor permeable and basically liquid impermeable. Polyurethane film having an adhesive on its undersurface sold by 3M under the designation 9841 is particularly suitable. Another material that can be used for the film with the adhesive on its undersurface is Hytrel polyester elastomer from E.I. DuPont de Nemours Company. The adhesive 30 on the undersurface of the cover 28 is an acrylate pressure sensitive adhesive that is suitable for skin contact. Its thickness is preferably in the range of 0.0004 inches to 0.004 inches, and most preferably in the range of 00007 inches-0.0013 inches.

The cover 28 with its adhesive 30 is moisture vapor permeable. In particular, it preferably transmits moisture at a rate greater than 300 grams per square meter per day (i.e., has a moisture vapor transmission rate or MVTR of at least 300). The cover 28 is arranged to be secured over a wound in a fashion such that a vacuum can be applied to the wound from the vacuum pump. To that end, the adhesive 30 seals the cover 28 to the skin around the wound, thereby forming a confined space under the cover and contiguous with the wound. The cover is preferably leak-proof, although small leak(s) can be tolerated. In particular, ideally a leak should be no greater than 20 percent of the flow capacity of the pump. A leak of up to 50% of the flow capacity of the pump can be tolerated, but is not ideal.

The cover 28 preferably is arranged to maintain its sealing state generally for up to 3 days or longer for typical suction assisted wound treatment applications. The moisture vapor permeability of the cover 28 allows moisture from the skin to be transmitted through the cover. If such moisture was not transmitted its build-up could cause the adhesive to become too wet and so that the cover might not maintain a sufficient seal. Also, the skin could become macerated.

Another critical property of the cover is that it needs to be very conformable to anatomical surfaces. A very thin, flexible conformable cover with adhesive is ideal for sealing a wound. When applying the cover to wounds, wrinkles often occur in the cover because it is being placed onto irregular surfaces. A very thin cover is desirable because the wrinkles can seal themselves (because of the adhesive). A thin cover is also desirable because the edge of the cover does not stick out beyond the skin surface. Thick edges lend themselves to getting rubbed off. Thin covers also have good moisture vapor permeability.

The releasably securable liner 32 will be described shortly. Suffice it for now ton state that it is provided to protect the adhesive 30 of the cover 28 until the adhesive is ready for application to the wound. As mentioned above, the releasably securable liner also works in conjunction with the stiffener 34 to facilitate the placement of the cover onto the wound.

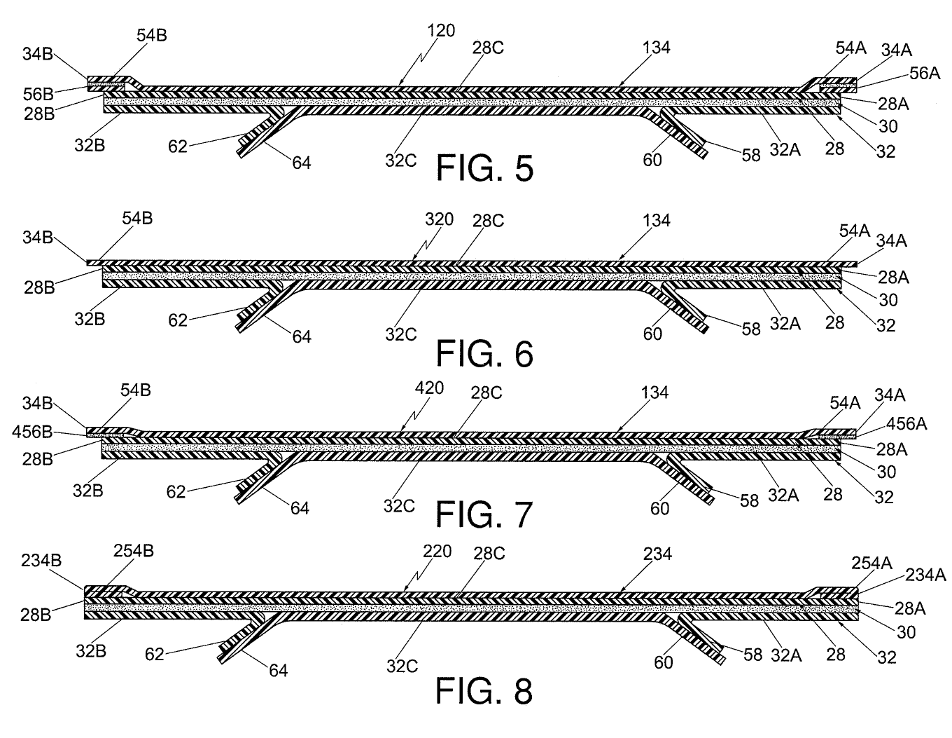

The stiffener 34 serves to provide some rigidity to the extremely flexible cover 28 once the releasably securable liner 32 has been removed from the composite structure 20. The stiffener 34 is in the form of a flexible sheet or layer that either alone or in combination with the cover 28, provides sufficient rigidity to the cover after the liner sheet 32 has been removed to enable the cover to be effectively secured to the wound, e.g., to keep the cover from wrinkling during application. To that end, the central portion of the stiffener, which constitutes its principal portion, is releasably secured to the principal portion of the cover 28 of the composite structure 20. The stiffener has an opposed pair of marginal edges 34A and 34B located on respective sides of the central or principal portion 34C, an upper surface and an undersurface. In the embodiment shown in FIG. 3 the width of the stiffener, i.e., the distance separating the marginal edges 34A and 34B is the same as the width of the cover 28, i.e., the distance separating the marginal edges 28A and 28B. In the embodiment of the composite structure shown in FIG. 5, which will be described later, the marginal edges of the stiffener extend beyond the marginal edges of the cover. In either case the portions of the stiffener 34 contiguous with its marginal edges 34A and 34B form a pair of respective handles 54A and 54B. These handles can take several forms. For example, in the embodiment shown in FIGS. 3-5, each handle is established by a respective adhesive tape strip. In particular, one adhesive tape strip 56A is secured along the undersurface of the stiffener 34 contiguous with the marginal side edge 34A and with one side of the central portion 34C to form the handle 54A. In a similar manner, the other adhesive tape strip 56B is secured along the undersurface of the stiffener contiguous with the marginal side edge 34B and with the opposite side of the central portion 34C to form the handle 54B. In addition to forming the handles 54A and 54B, the tape strips 56A and 56B, respectively, create respective finger spaces between them and the underlying marginal edge portion of the cover 28. Each of the finger spaces is adapted to receive one or more fingers of the user to facilitate grasping of the handles to effect the removal of the stiffener, as will be described later. As mentioned earlier, the portion of the stiffener located between the handles 54A and 54B defines the heretofore identified central or principal portion 34C and is the portion of the stiffener which is releasably secured to the corresponding central or principal portion of the underlying cover 28.

The stiffener 34 serves to keep the cover 28 from wrinkling and sticking to itself during application to a patient, yet it is thin enough that it easily conforms over complex patient anatomy. Once the cover is adhesively secured to the patient, the stiffener can be removed from the cover, leaving the remaining polyurethane film and adhesive that is very conformable and sealable around the wound.

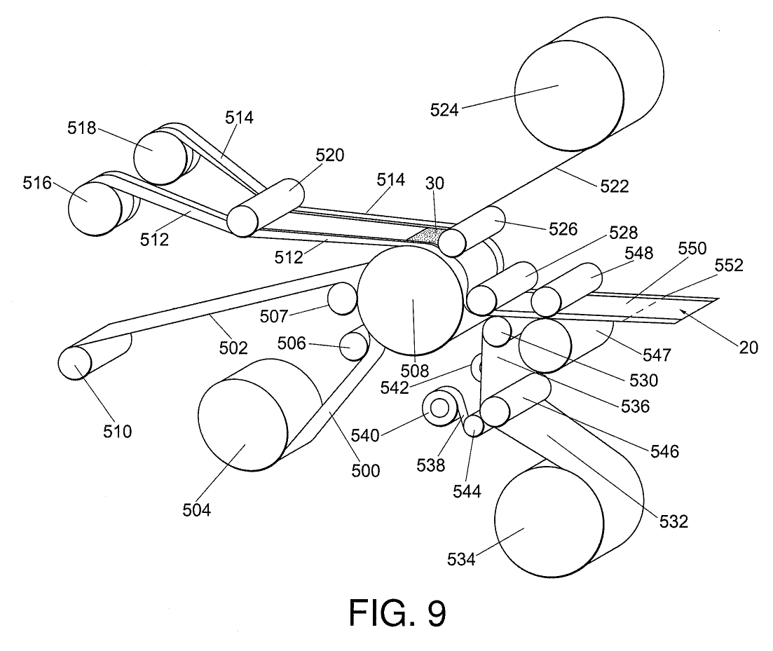

The stiffener 34 can be formed of any suitable material. One particularly suitable material is ethylene vinyl acetate (EVA) film, with 5% vinyl acetate content. Other materials contemplated for the stiffener are polyester film, polyethylene film, paper with an EVA coating and others. The thickness of the exemplary EVA stiffener can be in the range of 0.0005 inches-0.010 inches, with a preferred range of 0.0015 inches-0.0045 inches thick and a most preferred thickness of approximately 0,00225 inch. Moreover, the preferred EVA film is anisotropic, i.e., its undersurface has a very smooth finish (e.g., it has an Arithmetic Mean Roughness Value (Rₐ) of approximately 40 microinches, measured using a Hommel Tester T500), while its upper surface has a somewhat rougher or matte finish (e.g., it has an Arithmetic Mean Roughness Value (Rₐ) of approximately 140 microinches, measured using a Hommel Tester T500). This construction ensures that the smooth undersurface of the EVA stiffener is amenable to being releasably thermally bonded to the polyurethane film making up the cover, while the matte upper surface of the stiffener is resistant to such bonding. In particular, as mentioned above, the central or principal portion of the undersurface of the stiffener 34 is releasably secured to the corresponding principal portion of the upper surface of the cover by thermally laminating the layers together. That action can be accomplished as shown in FIG. 9 (and which will be described later) by providing a continuous web of polyurethane film having the parameters described above and a continuous web of EVA having the parameters described above and laminating them together at a temperature in the range of approximately 160° F.-185° F., with a pressure of approximately 8-13 PLI (pounds per linear inch) and at a lamination speed in the range of approximately 4 feet/minute to 12 feet/minute. Other combinations could also work. The resulting releasable bond strength between the polyurethane film cover 28 and the EVA stiffener 34 is less than 1 oz per inch width when measured with 180 degree peel test with a pull speed of 10 in/minute. Preferably the bond strength is less than 0.5 oz/inch and is most preferably in the range of 0.01-0.3 oz/inch when measured with the aforementioned peel test and pull speed.

The tape strips 56A and 56B serve as separators to keep the marginal edges of the stiffener 34 from bonding to the underlying corresponding marginal edge portions of the polyurethane film cover 28. Moreover, as described above each also acts as a handle or tab that can be grasped once the cover is applied to the patient. The stiffener can then be removed from the cover by use of the handles. Each of the tape strips is secured to the undersurface of the stiffener by the adhesive of the tape strip to render each handle of the stiffener a little stiffer or more rigid than the principal portion of the stiffener. The paper layer of each of the tape strips provides a little thickness that makes the handle or tab easy to grasp by the user. The tape can be of any suitable material. One example of a paper tape that can be used is Shurtape CP 64, available from Shurtape Technologies, having a thickness of 0.0064 inches. Numerous other tapes, paper or otherwise, could work.

In FIG. 5 there is shown an alternative embodiment of a composite structure 120 constructed in accordance with this invention. The structure 120 is identical in construction to the composite structure 20 except that the marginal edge portions of its stiffener 134, i.e., the marginal edge portions forming the handles, extend outward beyond the marginal edges of the cover 28. In the interest of brevity the common components of the composite structure 120 will be given the same reference numbers as those of the embodiment of the composite structure 20 shown in FIG. 3 and the details of the construction and operation of those components will not be reiterated. Thus, as can be seen the composite structure 120 basically comprises the heretofore identified cover 28 and the heretofore identified liner 32. The stiffener of the composite structure 120 is designated by the reference number 134. The marginal edges 34A and 34B of the stiffener 134 extend slightly beyond the respective marginal edges 28A and 28B of the cover 28. The tape strips 56A and 56B are secured under those marginal edge portions of the stiffener so that portions of the respective tape strips also extend beyond the marginal edge of the cover. Accordingly, it may be somewhat easier to grasp the handles 54A and 54B of the stiffener of the composite structure 120 of FIG. 5 as compared to the composite structure 20 of FIG. 3 when the stiffener is removed from the cover (as will be described later).

In FIG. 8 there is shown an alternative embodiment of a composite structure 220 constructed in accordance with this invention. The composite structure 220 is identical to the structure 20, except for the stiffener 234 (to be described shortly). In the interest of brevity, the common components of the composite structure 220 will be given the same reference numbers as those of the embodiment of the composite structure 20 shown in FIG. 3 and the details of the construction and operation of those components will not be reiterated. Thus, as can be seen the composite structure 220 basically comprises the heretofore identified cover 28 and the heretofore identified liner 32. Each of the marginal edges 234A and 234B of the stiffener 232 is folded under itself to form a respective handle 254A and 254B instead of using tape strips on the undersurface of the stiffener as is the case of stiffener 34 of composite structure 20. Since the upper surface of the stiffener 234A has a matte finish, the portion of that surface which forms the handle 254A will be juxtaposed over the upper surface of the marginal edge 28A of the cover. In a similar manner the portion of the matte surface which forms the handle 254B will be juxtaposed over the upper surface of the marginal edge 28B of the cover. Accordingly, when the webs of material forming the stiffener 232 and cover 28 are laminated together in a similar manner to that described above the central or principal portion of the stiffener 234 will be releasably thermally bonded to the corresponding principal portion of the upper surface of the cover, while the handle/tabs 254A and 254B remain separated from the corresponding underlying portions of the cover. In particular, the matte surface of the underside of each of the tab/handles does not bond to the upper surface of the cover, thereby creating respective marginally located finger-receiving spaces located between the stiffener and cover.

Another way to keep the peripheral edge of the stiffener separate from the peripheral edge of cover is to thermally bond only the central or principal portion of the stiffener to the central or principal portion of the cover. This could be done without using many separator (e.g., tape strips or folded portions of the stiffener). The embodiment of composite structure 320 shown in FIG. 6 constitutes a variation of composite structure 120 and is an example of such an arrangement. The common components of the composite structure 320 to the composite structure 120 are given the same reference numbers and the details of their construction and operation will not be reiterated in the interest of brevity. Thus, as can be seen the stiffener 134 does not include any tape strip 56A and 56B on its undersurface at its marginal edges. Instead only the central or principal portion of the stiffener 134 is releasably thermally bonded to the central or principal portion of the cover 28. It should be understood that the principal portion of the cover does not extend to the marginal edge of the cover, i.e., there is a pair of band areas of the cover contiguous with the peripheral edges of the cover that are not bonded to the stiffener.

In FIG. 7 there is shown another alternative embodiment of a composite structure 420 constricted in accordance with this invention. The composite structure 420 is identical in construction to the composite structure of FIG. 6 except for the use of two bands of silicone coating on the undersurface of the stiffener in place of the tape strips 56A and 56B. In the interest of brevity the common components of composite structure 420 and composite structure 120 will be given the same reference numbers and the details of their construction and operation will not be reiterated. Thus, as can be seen, a thin band of silicone 456A is coated on the undersurface of the stiffener 134 contiguous with its marginal edges 34A and a similar thin band of silicone 456B is coated on the undersurface of the stiffener contiguous with the other marginal edge 34B. The central or principal portion of the stiffener, i.e., the portion between the silicone bands, is releasably thermally secured to the corresponding principal portion of the cover 28 disposed therebelow.

The details of the release liner 32 will now be described. In accordance with a preferred aspect of this invention the release liner 32 is provided in three separate sections 32A, 32B and 32C, each of which serves to protect a respective portion the adhesive 30 of the cover 28 prior to use of the cover. The release liners 32A-32C also permit secure handling and maneuvering of the cover when applying it over the wound bed. The release liner sections 32A-32C are similar to those disclosed in the aforementioned U.S. Pat. No. 4,614,183 (McCracken et al.), and those sections are each formed of a material that is well known in the industry, e.g., siliconized paper. The silicone coating on the paper forming each section 32A-32C prevents that section from permanently sticking to the adhesive 30 of the cover. Paper is just one material that can be used as a release liner section. Siliconized polyethylene, such as the material designated Poly Slik by Loparex, Inc. of Willowbrook, Ill., can also be used as the material making up the release liner sections. In the embodiments shown the release liner section 32A is located on the portion of the adhesive 30 contiguous with the marginal edge 28A. The release liner section 32B is located on the portion of the adhesive 30 contiguous with the marginal edge 28B and the release liner section 32C is located on the portion of the adhesive 30 contiguous with the central portion of the cover. The inner marginal edge of section 32A is in the form of a flange 58. The adjacent marginal edge of the central section 32C is in the form of a flange 60, which abuts the flange 58 of the section 32A. In a similar manner, the inner marginal edge of section 32B is in the form of a flange 62. The adjacent marginal edge of the central section 32C is in the form of a flange 64, which abuts the flange 62 of the section 32B. The flanges serve as portions of the liner sections which can be grasped between the fingers of the user to remove them from the undersurface of the cover when the cover is to be placed onto the wound as will be described later. It should be pointed out at this juncture that the portions of the abutting flanges are shown in the figures of this application as projecting at a substantial acute angle to the plane of the composite structure. In fact, those flanges are normally disposed folded or flattened down somewhat when the composite structure is stored for future use to conserve space, i.e., to enable a plurality of structures 320 to be stacked up. In use the folded or flattened down flanges can then be unfolded to assume an orientation similar to that shown, whereupon each flange can be individually grasped to effect the removal of its associated liner section as will be described momentarily.

The application of the composite structures of this invention to a wound is carried out in the following manner. Just prior to application the central section 32C of the release liner is removed from the composite structure by grasping either of its flanges 60 or 64 and peeling it off of the adhesive 30 on the underside of the cover 28, thereby exposing the central portion of the adhesive. The composite structure can then be grasped between the user's fingers at each marginal edge portion, i.e., the portion made up of a handle and the underlying marginal edge portion of the liner section 32A or 32B. The composite structure can then be maneuvered to the desired position over the wound. The exposed portion of the adhesive 30 of composite structure is then be brought into engagement with the wound and the top surface of the stiffener smoothed over with a hand to secure the central portion of the cover to the wound without any wrinkles. The presence of the stiffener ensures that the central portion of the thin flexible cover whose adhesive is exposed as a result of the removal of liner section 32C doesn't wrinkle or stick to itself. The release liner sections 32A and 32B can then be removed by grasping their respective flanges 58 and 62 and peeling the liner sections out from under the cover 28. Once the release liner sections 32A and 32B have been peeled away from their respective regions of the cover, the top surface of the stiffener over those regions of the cover can be smoothed over by the hand of the user to secure those portions of the cover to the wound with any wrinkles. The presence of the stiffener over those regions of the cover also ensures that the side portions of thin flexible cover whose adhesive is exposed as a result of the removal of liner sections 32A and 32B doesn't wrinkle or stick to itself. The stiffener 34 can then be removed by peeling it off of the cover 28 via either of its handles. The tube attachment device 24 can then be secured to the cover 28 to connect the cover to the suction assisted wound treatment system 10, so that suction can be applied to the wound below the cover 28.

In accordance with one preferred embodiment of this invention the releasable bond strength between the adhesive 30 on the undersurface of the polyurethane film making up the cover 28 and the siliconized liner sheets 32A-32C are in the range of approximately 0.35-0.65 oz per inch width when measured with 180 degree peel test with a pull speed of 10 in/minute.

The production of a composite structure 20, like shown in FIG. 3, will now be discussed with reference to FIG. 9 using the apparatus or system shown therein. To that end, a continuous web 500 made up of a layer of polyurethane film having an adhesive on its undersurface (like that disclosed above to form the cover 28 and adhesive 30) and a layer in the form of releasable waste liner 502 (e.g., a web of siliconized paper) is provided from a roll 504. The web 500 is fed between a pair of nip rollers 506 and 507 and a drum 508, whereupon the web of waste liner 502 is peeled away and reeled up on a take-up reel 510. The web of the polyurethane film with the adhesive then passes about a portion of the periphery of drum 508, with the adhesive side of the web of film being exposed. A pair of narrow, continuous webs 512 and 514 of release liner material, e.g., siliconized paper, are provided from respective supply rolls 516 and 518. The webs 512 and 514 form the heretofore identified release liner sections 32A and 32B of the composite structure 20. To that end, the inner marginal edge of each of the webs 512 and 514 is folded over itself. The folded over portions form the respective flanges 58 and 62 of the release liners 32A and 32B, respectively. The webs 512 and 514 with their siliconized surfaces facing downward, and with their folded over portions facing upward, pass under a guide or pressure roller 520 to form a sharp fold in each web. From there the webs 512 and 514 are brought into engagement with the exposed adhesive of the polyurethane film web on the drum 508, whereupon the two webs 512 and 514 are releasably secured to the web of polyurethane film along the two marginal edges thereof. This action leaves the adhesive 30 on the central or principal region of the polyurethane film exposed. A wider, continuous web 52 of release liner material, e.g., siliconized paper, is provided from a supply roll 524. The web 522 forms the heretofore identified central release liner section 32C. To that end, the central web 522 with its siliconized surface facing downward is passed under a guide roller 526 which presses it into engagement with the exposed adhesive 30 of the polyurethane web on the drum 508. Each of the marginal edges of the central web 522 overlies the adjacent folded marginal edge portion of a respective one of the previously placed release liner webs 512 and 514, whereupon those overlying portions form the heretofore identified flanges 60 and 64 of the central release liner section 32C. It should be pointed out at this juncture that the webs 512 and 514 can be provided as prefolded webs from the supply rolls 516 and 518 or may be provided as flat webs which are folded (formed) by a former (not shown) during manufacturing by the apparatus shown in FIG. 9.

The web of polyuethane film with the three release liner webs now secured to its adhesive 30 is then passed between an opposed pair of pressure applying rollers 528 and 530. It is at these rollers that a web of continuous material, e.g., EVA, forming the stiffener is brought for releasable securement to the web of polyurethane film. In particular, a continuous web 532 of EVA material like that described previously is provided from a supply roll 534, with the matte side 536 of the web 532 facing upward and the smooth side facing downward. A pair of narrow, continuous webs 538 of adhesive tape are provided from respective supply rolls 540 and 542. The webs 538 form the heretofore identified separator strips 56A and 56B of the composite structure 20. To that end, the tape strips 56A and 56B, with their adhesive surface facing downward are carried about the surface of a guide roller 544 and into engagement with the smooth surface on the underside of the web 532 to adhesively secure the tape strips to the web 532. Pressure to effect the securement of the tape strips 538 to the smooth surface of the web 532 is provided by an opposed pressure applying roller 546. The web 532 forming the stiffener (the “stiffener forming web”), with the adhesive tape separator strips 538 now secured to its smooth surface is brought to the roller 530, with the tape strips 538 facing upward. That web is then juxtaposed under the polyurethane web the liner webs 512, 522 and 514 releasably secured on its adhesive 30 (the “cover forming web”). The two juxtaposed webs are brought between a heated roller 547 and a pressure applying or backing roller 548, whereupon the central or principal portion of the stiffener forming web is thermally laminated to the central or principal portion of the cover forming web in a releasable bond. The separator strips 538 on the undersurface of the stiffener forming web prevent the underlying marginal edge portions of the stiffener forming web from being secured to the cover forming web when the releasable bond between the principal portions of the stiffener forming web and the cover forming web is produced. The resulting composite web 550 can then be cut into sequential sheets at a cutting or station (not shown) along a transverse cut line 552 to form a series of composite series 20.

It should be pointed out at this juncture that the method as just described and the apparatus for producing the composite structure can be modified to produce other composite structures in accordance with this invention. For example, to manufacture the composite structure 220 shown in FIG. 8 in lieu of providing adhesive tape separator strips 538, the marginal edges of the stiffener forming web 532 can be folded under itself. Thus, the web 532, which passes over roller 530 has the matte surface of its folded marginal edge portions facing upward. By so doing those matte surfaces are brought into abutment with the cover forming polyurethane film web. Since the matte finish of the web 532 is resistant to thermal bonding, the only portion of the abutting webs that will be thermally laminated together is the central or principal portions, leaving the marginal edge portions of the stiffener free of the corresponding marginal edge portions of the cover, thereby forming the heretofore identified handles.

It also should be pointed out that composite structures constructed in accordance with this invention can be of any size. In addition, the composite structure may be cut by a scissors or any other cutting instrument to tailor the size to a desired size for use on a particular wound for some other purpose, e.g., a drape. Moreover, a composite structure may be made in accordance with this invention comprising only a single handle (e.g., a single tape on the underside of one marginal edge of the stiffener) and only two release liner sections disposed beside each other. That embodiment can be made by cutting any of the embodiments of the composite structures described heretofore along a longitudinal center line between the opposed marginal edges of the structure.

The composite structures of this invention offer various advantageous features. For example, the stiffener can be removed in one step by a user, without leaving any thickened edge on the polyurethane film that has to be removed, e.g., torn away along a perforated line (such as has characterized some of the prior art). As will be appreciated by those skilled in the art tearing away a thickened edge of the polyurethane film can disrupt its adhesive seal. Moreover, if that thicker edge of the polyurethane film isn't removed it can be prone to accidental peeling since it is thicker than the film itself.

The use of the finger space between the stiffener and the cover at the marginal edge of the composite structure also is instrumental in minimizing the chance of disturbing the cover after it has been adhesively secured to the wound, but before the stiffener has been removed. In this connection, the finger space isolates the marginal edge of the cover from the stiffener. Thus when one peels the stiffener off of the cover, the peeling force is not applied to the marginal edge of the cover (which is adhered to the skin), thereby decreasing the chance of accidentally causing the cover to be peeled off of the skin when the stiffener is removed.

The use of a three section release liner in combination with a stiffener having its handles/separator strips secured to the undersurface of the stiffener facilitates easy and effective placement of the cover on the wound. In particular, the outer-located (side) release liners 32A and 32B hold the polyurethane film of the cover 28 in place until the user applies the cover. This helps keep the marginal edges of the cover (which are free of the corresponding marginal edges of the stiffener, i.e., there is the heretofore discussed finger space therebetween) from wrinkling prior to application.

The bond between the adhesive 30 on the polyurethane film of the cover 28 and the siliconized release liners can be stronger than the releaseable bond between the stiffener and the polyurethane film of the cover, yet still result in an easy to use, yet effective composite for adhesive securement to a wound or for other applications.

Without further elaboration the foregoing will so fully illustrate our invention that others may, by applying current or future knowledge, adopt the same for use under various conditions of service.

What is claimed is:

1. A composite structure comprising a cover, a stiffener, and a releasable liner, said cover being arranged for use on a wound of a patient and being formed of a very thin, flexible film having an upper surface, an undersurface, a principal portion, a pair of opposed marginal edges, and an adhesive on said undersurface of said film, said stiffener comprising a sheet having an upper surface, an undersurface, a principal portion, a pair of opposed marginal edges and at least one handle, said at least one handle being more rigid than said principal portion of said stiffener and forming a portion of said undersurface of said stiffener contiguous with a marginal edge of said stiffener, said stiffener being disposed over said cover, with said undersurface of said stiffener at said principal portion of said stiffener being releasably secured to said upper surface of said cover at said principal portion of said cover, said at least one handle defining between it and the marginal edge of said cover disposed therebelow a finger space in which a finger of a user can be inserted to separate said stiffener from said cover, said releasable liner comprising at least one section releasably secured to said adhesive to protect said adhesive and being removable when desired to expose said adhesive, whereupon said cover can be adhesively secured over the wound by said adhesive, said stiffener and said at least one handle being removable as a unit from said cover.

2. The composite structure of claim 1 wherein said releasable liner comprises at least two sections, each of said sections including a flange portion arranged to be grasped to enable said section to be removed from said adhesive.

3. The composite structure of claim 2 wherein said releasable liner comprises three laterally located sections in the form of a central section and two side sections, said central section having a pair of sides, each of said side sections being disposed beside a respective one of said sides of said central section.

4. The composite structure of claim 1 wherein said under surface of said stiffener is thermally releasably secured to said upper surface of said cover.

5. The composite structure of claim 1 wherein said handle comprises a strip of material.

6. The composite structure of claim 4 wherein said strip of material comprises a tape strip adhesively secured to said undersurface of stiffener contiguous with said marginal edge of said stiffener.

7. The composite structure of claim 1 comprising a pair of handles, each of said handles forming a respective portion of said undersurface of said stiffener at a respective marginal edge of stiffener, and wherein said undersurface of said stiffener is releasably secured to said upper surface of said cover between said handles.

8. The composite structure of claim 7 wherein said undersurface of said stiffener is releasably thermally secured to said upper surface of said cover between said handles.

9. A composite structure comprising a cover and a stiffener, said cover being arranged for use on a wound of a patient and being formed of a very thin, flexible film having an upper surface, an undersurface, a principal portion, at least one marginal edge portion and an adhesive on said undersurface of said film, said at least one marginal edge portion of said cover and said principal portion of said cover being of essentially the same flexibility as each other, said stiffener comprising a sheet having an upper surface, an undersurface, a principal portion and at least one marginal edge portion, said stiffener being disposed over said cover with said undersurface of said stiffener at said principal portion of said stiffener being releasably secured to said upper surface of said cover at said principal portion of said cover, and wherein said at least one marginal edge portion of said stiffener is separated from said at least one marginal edge portion of said cover disposed therebelow by a finger space.

10. The composite structure of claim 9 wherein said undersurface of said stiffener at said principal portion of said stiffener is thermally releasably secured to said upper surface of said cover at said principal portion of said cover.

11. The composite structure of claim 9 wherein said undersurface of said at least one marginal edge portion of said stiffener comprises a strip forming a handle for said stiffener.

12. The composite structure of claim 11 wherein said strip comprises a tape strip adhesively secured to said undersurface of at least one of said marginal edge portions of said stiffener.

13. The composite structure of claim 9 additionally comprising a releasable liner.

14. The composite structure of claim 13 wherein said releasable liner comprises at least two sections, each of said sections including a flange portion arranged to be grasped to enable said section to be removed from said adhesive.

15. The composite structure of claim 14 wherein said releasable liner comprises three laterally located sections in the form of a central section and two side sections, said central section having a pair of sides, each of said side sections being disposed beside a respective one of said sides of said central section.

16. The composite structure of claim 11 wherein said at least one marginal edge portion of said stiffener having said finger space therebelow forms a handle, said handle being stiffer said principal portion of said stiffener.

17. A suction assisted wound care system comprising a source of suction, a coupling member, and a composite structure, said composite structure comprising a cover adapted to be adhesively secured over a wound of a patient to create a confined space to which a vacuum can be applied, a stiffener and a releasably securable liner, said coupling member being arranged to be coupled to said source of suction for applying suction to said confined space, said cover being formed of a very thin, flexible film having an upper surface, an undersurface, a principal portion, at least one marginal edge and an adhesive on said undersurface of said film arranged to secure said cover to the wound, said stiffener comprising a sheet having an upper surface, an undersurface, a principal portion, at least one marginal edge and at least one handle, said at least one handle forming a portion of said undersurface of said stiffener contiguous with said at least one marginal edge of said stiffener, said stiffener being disposed over said cover, with sad undersurface of said stiffener at said principal portion of said stiffener being releasably secured to said upper surface of said cover at said principal portion of said cover, said at least one handle defining between it and said at least one marginal edge of said cover disposed therebelow a finger space in which a finger of a user can be inserted, said releasable liner comprising at least one section releasably secured to said adhesive to protect said adhesive and being removable when desired to expose said adhesive, whereupon said cover can be adhesively secured over the wound by said adhesive, said stiffener and said at least one handle being removable as a unit from said covers.

18. The system of claim 17 wherein said system additionally comprises a wound packing having a wound contact surface adapted to engage the wound of the patient, said wound packing being arranged to be located within said confided space with said wound contact surface engaging the wound.

19. The system of claim 17 wherein said system additionally comprises a receptacle coupled to said source of suction and adapted for receiving fluid from said wound.

20. The system of claim 17 wherein said principal portion of said cover and the portion of said cover contiguous with said at least one marginal edge are of essentially the same flexibility as each other.

21. The system of claim 17 wherein said at least one handle is stiffer than said principal portion of said stiffener.

22. A method of providing suction assisted wound treatment to a wound of a patient comprising:
providing a composite structure comprising a cover, a stiffener and a releasably securable liner, said cover being formed of a very thin, flexible film having an upper surface, an undersurface, a principal portion, at least one marginal edge and an adhesive on said undersurface of said film arranged to secure said cover to the patient, said stiffener comprising a sheet having an upper surface, an undersurface, a principal portion, at least one marginal edge and at least one handle, said at least one handle forming a portion of said undersurface of said stiffener contiguous with said at least one marginal edge of said stiffener, said stiffener being disposed over said cover, with said undersurface of said stiffener at said principal portion of said stiffener being releasably secured to said upper surface of said cover at said principal portion of said cover, said at least one handle defining between it and said at least one marginal edge of said cover disposed therebelow a finger space in which a finger of a user can be inserted, said releasable liner comprising at least one section releasably secured to said adhesive to protect said adhesive and being removable when desired to expose said adhesive;
removing said at least one section of said releasable liner from said cover to expose said adhesive and applying said cover over the wound to: produce a confined space to which suction can be applied;
grasping said at least one handle of said composite: structure via said finger space to remove said stiffener and said at least one handle as a unit from said cover; and
coupling a source of suction to said confined space.

23. The method of claim 22 additionally comprising applying a wound packing having a wound contact surface to the wound so that said wound packing is located within said confined space, with said wound contact surface engaging the wound.

24. A method of treating a wound of a patient comprising:
providing a composite structure comprising a cover, a stiffener and a releasably securable liner, said cover being formed of a very thin, flexible film having an upper surface, an undersurface, a principal portion, at least one marginal edge and an adhesive on said undersurface of said film arranged to secure said cover over the wound, said at least one marginal edge of said cover and the portion of said cover contiguous therewith being of essentially the same flexibility as said principal portion of said cover, said stiffener comprising a sheet having an upper surface, an undersurface, a principal portion, at least one marginal edge and at least one handle said at least one handle forming a portion of said undersurface of said stiffener at said at least one marginal edge of said stiffener, said undersurface of said stiffener at said principal portion of said stiffener being releasably secured to said upper surface of said cover at said principal portion of said cover, said at least one handle defining between it and said at least one marginal edge of said cover disposed therebelow a finger space in which a finger of a user can be inserted, said releasable liner comprising at least one section releasably secured to said adhesive to protect said adhesive and being removable when desired to expose said adhesive;
removing said at least one section of said releasable liner from said cover to expose said adhesive and applying said cover on said wound; and
grasping said at least one handle of said composite structure via said finger space to remove said stiffener and at least one handle as a unit from said cover.

25. The method of claim 24 additionally comprising applying a wound packing having a wound contact surface to the wound so that said wound packing is located between said cover and the wound with said wound contact surface in contact with the wound.

26. A method for making a composite structure including a cover for adhesive application to the body of a patient, said method comprising:
providing a cover formed of a very thin, flexible film having an upper surface, an undersurface; a principal portion, a pair of opposed marginal edges and an adhesive on said undersurface of said film;
providing a stiffener comprising a sheet having an upper surface, an undersurface, a principal portion and a pair of opposed marginal edges;
providing a strip to said undersurface of said stiffener immediately adjacent one of said marginal edges of said stiffener to form at least one handle, said at least one handle being stiffer than said principal portion of said stiffener; and
juxtaposing said stiffener and said cover to releasably secure said undersurface of said stiffener at said principal portion of said stiffener to said upper surface of said cover at said principal portion of said cover.

27. The method of claim 26 wherein said method comprises releasably thermally securing said undersurface of said stiffener at said principal portion of said stiffener to said upper surface of said cover at said principal portion of said cover.

28. The method of claim 26 comprising applying a liner sheet to said adhesive.

29. The method of claim 28 wherein said liner sheet comprises three laterally located sections in the form of a central section and two side sections, said central section having a pair of sides, each of said side sections being disposed beside a respective one of said sides of said central section.

30. The method of claim 26 wherein said cover sheet is in the form of a continuous cover sheet forming web, and wherein said liner sheet is in the form of a continuous liner sheet forming web, said cover sheet forming web being supported to expose said adhesive on the undersurface thereof, said liner sheet forming web being brought into engagement with said adhesive on said cover sheet forming web to releasably secure said liner sheet forming web to said cover sheet forming web.

31. The method of claim 30 wherein said liner sheet forming web comprises three individual continuous webs, each of said three continuous webs forming a respective section of said liner sheet.

32. A method for making a composite structure including a cover for adhesive application to the body of a patient, said method comprising:
providing a cover formed of a thin, flexible film having an upper surface and an undersurface, said undersurface of said cover having an adhesive thereon;
disposing said cover directly on a support surface, wherein said upper surface of said cover is in direct engagement with said support surface and said adhesive on said undersurface of said cover is exposed:
applying a release liner directly to said adhesive on said undersurface of said cover to releasably secure said release liner thereto;
providing a stiffener comprising a sheet having an upper surface and an undersurface; and
releasably laminating said undersurface of said stiffener to said upper surface of said cover.

33. The method of claim 32 wherein said support surface comprises the outer surface of a drum, and wherein said cover is in the form of a continuous cover forming web having said upper surface and said undersurface with said adhesive thereon, said method comprising disposing said cover forming web directly on said outer surface of said drum, with said upper surface of said cover forming web in direct engagement with said outer surface of said drum.
